(19) United States
(12) Patent Application Publication
(10) Pub. No.: US 2024/0240123 A1
(43) Pub. Date: Jul. 18, 2024

(54) TISSUE DISSOCIATION USING INTERNALLY THREADED TUBES

(71) Applicant: OMNI INTERNATIONAL, INC., Kennesaw, GA (US)

(72) Inventors: Rodney NASH, Tucker, GA (US); Zachary MOREHOUSE, Tampa, GA (US); Paul H. ROBERTS, Kennesaw, GA (US); William Z. TODD, Kennesaw, GA (US); Peter TORTORELLI, Marietta, GA (US); Jason PAULSEL, Peachtree City, GA (US); John D. BUNTING, Roswell, GA (US); Gabriella RYAN, Atlanta, GA (US)

(73) Assignee: OMNI INTERNATIONAL, INC., Kennesaw, GA (US)

(21) Appl. No.: 18/410,533

(22) Filed: Jan. 11, 2024

Related U.S. Application Data
(60) Provisional application No. 63/438,854, filed on Jan. 13, 2023.

(51) Int. Cl. C12M 1/12; C12M 1/00

(52) U.S. Cl. CPC C12M 23/06; C12M 23/40

(57) ABSTRACT

A laboratory processing tube assembly mounts to a processing unit for dissociating an organically derived sample. The tube assembly includes a tube and a cap that form a tube chamber that contains the sample during dissociation. The tube includes at least one internal thread extending inwardly from and peripherally about its inner surface in a helical arrangement, with the internal helical thread including a ridge defining two impact surfaces facing in generally opposite directions, and with a first one of the impact surfaces impacted by the sample as the sample flows in a first axial direction in the tube chamber and a second one of the impact surfaces impacted by the sample as the sample flows in a second opposite axial direction in the tube chamber during axially-reciprocal tube-assembly motion to dissociate the sample.

### CROSS-REFERENCE TO RELATED APPLICATION

This application claims the priority benefit of U.S. Provisional Patent Application Ser. No. 63/438,854, filed Jan. 13, 2023, which is hereby incorporated herein by reference.

### TECHNICAL FIELD

The present disclosure relates generally to laboratory devices and methods for processing sample materials, and particularly to tubes for holding samples during processing to dissociate tissue cells.

### BACKGROUND

In the life sciences, a common need is dissociation of tissue samples with an output being single cells that have been preserved as intact cells for downstream uses, with these output single intact cells typically but not always necessarily viable. These samples can be for example tissue (e.g., liver), fluid (e.g., blood), or bone of a human or other animal, another organic material, or another substance. These output single intact cells are needed for many downstream uses, including life-sciences applications such as biomedical research, cell therapies, cultures, identification, and diagnostics, as well as sorting for these and other downstream processes (e.g., DNA and RNA detection and analysis). Thus, sample dissociation is an important step at the front end of nearly all workflows that use single intact cells.

Current techniques for sample dissociation into single intact cells include enzymatic dissociation and mechanical dissociation, with mechanical disassociation being either manual (human-powered use of handheld unpowered tools such as surgical utensils or Dounce homogenizers) or automated (using electric-powered devices with internal blades and/or magnets).

But these techniques all have drawbacks. Manual mechanical dissociation is the traditional technique. With this technique, the surgical utensils are typically scalpels, blades, or tweezers used to process a sample held in a petri dish. And Dounce homogenizers are handheld mortar-and-pestle implements. In both these types of mechanical manual disassociation, the output is variable and highly dependent on the individual user/operator, because a person has to manually and vigorously apply input motions/forces (e.g., stirring or grinding) for an extended period of time (typically, about 20 minutes, depending on the person because some people are more efficient than others), because different people have different stirring/grinding techniques, and because the same person will stir/grind differently over an extended period of time (e.g., as they tire). So in practice, these manual implements produce results that are widely variable, very inconsistent, and highly unreliable. Also, Dounce homogenizers are very ineffective on samples including connected tissue, such as muscle.

Furthermore, automatic/powered devices with blades and magnets produce very aggressive dissociation that results in far fewer single intact cells, because the cells are damaged during the aggressive dissociation process. As such, these automatic devices are not suitable for use to produce sample dissociation when a large volume of output single intact cells is desired. Finally, enzymes tend to damage cells and alter the transcriptome of the cell, and thus they also are not suitable for use to produce sample dissociation when a large volume of output single intact cells is desired.

As such, all current sample dissociation techniques tend to produce results that are inconsistent, slow/time-consuming, and/or not efficient/effective for obtaining single intact cells from tissue samples.

### SUMMARY

Generally described, the present disclosure relates to a laboratory tube assembly that mounts to a processing unit for use to dissociate an organically derived sample by an oscillating tube-assembly motion including an axially-reciprocal component. In example embodiments, the tube assembly includes a tube and a cap that removably couple together to form a tube chamber that has a longitudinal axis and that contains the sample during dissociation, and the tube has a peripheral inner surface. In addition, the tube assembly includes at least one internal thread extending inwardly from and peripherally about the inner surface of the tube in a helical arrangement. The internal helical thread includes a ridge defining two impact surfaces facing in generally opposite directions at a non-perpendicular angle relative to the longitudinal axis of the tube chamber and relative to the inner surface of the tube. In use, a first one of the impact surfaces is impacted by the sample as the sample flows in a first axial direction in the tube chamber and a second one of the impact surfaces is impacted by the sample as the sample flows in a second opposite axial direction in the tube chamber during the axially-reciprocal tube-assembly motion to dissociate the sample.

In typical embodiments, the sample impacts against the impact surfaces of the internal peripheral thread during dissociation use and separates the sample into a dissociate of single intact cells, without using enzymes or magnetic forces in the tube chamber (for purely mechanical dissociation), or with using only mild enzymes in the tube chamber (for enzymatic assisted/boosted mechanical dissociation). The impact surfaces facing in directions that are angled from the longitudinal axis of the tube chamber can result in blunt impact forces on the sample and shearing forces on the sample during dissociation use. For example, the blunt impact forces on the sample can be greater in the first direction that in the second direction, and the shearing forces on the sample can be greater in the second direction that in the first direction, due to the internal helical thread producing a vortical angular flow of the sample.

In some embodiments, the internal helical thread forms a single continuous ridge. For example, the internal helical thread can define multiple (at least two) complete revolutions about the peripheral inner surface of the tube.

In some embodiments, the internal helical thread extends along 80 percent to 90 percent of an entire length of the internal peripheral surface of the tube and/or has a thread pitch of 15 threads per inch to 25 threads per inch. Also, in some embodiments the internal helical thread has a profile with a thread angle of 25 degrees to 75 degrees (so the oppositely facing impact surfaces are angled relative to each other) or has a square profile with a zero degree thread angle (so the oppositely facing impact surfaces are parallel to each other).

In some embodiments, the internal helical thread is formed on a sleeve that inserts into the tube and forms the peripheral inner surface of the tube. Also, in some embodiments the internal helical thread is formed by two ridges in a double helix arrangement.

Another example embodiment includes a laboratory dissociation system for dissociating a sample. In example embodiments, the system includes a processing unit that produces an oscillating tube-assembly motion including an axially-reciprocal component. The system also includes a tube assembly that mounts to the processing unit. The tube assembly includes a tube and a cap that removably couple together to form a tube chamber that has a longitudinal axis and that contains the sample during dissociation use. Additionally, the tube has a peripheral inner surface and at least one internal thread extending inwardly from and peripherally about the peripheral inner surface in a helical arrangement. The internal helical thread includes a ridge defining two impact surfaces facing in generally opposite directions at a non-perpendicular angle relative to the longitudinal axis of the tube chamber and relative to the inner surface of the tube. In use, a first one of the impact surfaces is impacted by the sample as the sample flows in a first axial direction in the tube chamber and a second one of the impact surfaces is impacted by the sample as the sample flows in a second opposite axial direction in the tube chamber during the axially-reciprocal tube-assembly motion to dissociate the sample.

In some embodiments, the laboratory processing unit is a bead mill homogenizer. In such embodiments, the tube-assembly motion produced by the bead mill homogenizer can be a swashing motion including the axially-reciprocal component.

Another example embodiment includes a laboratory dissociation method. In example embodiments, the method includes loading a sample and a liquid buffer into a tube assembly including a tube and a cap that removably couple together to form a tube chamber that has a longitudinal axis and that contains the sample during dissociation use. The tube has a peripheral inner surface and at least one internal thread extending inwardly from and peripherally about the inner surface in a helical arrangement. The internal helical thread includes a ridge defining two impact surfaces facing in generally opposite directions at a non-perpendicular angle relative to the longitudinal axis of the tube chamber and relative to the inner surface of the tube. The method further includes mounting the loaded tube assembly to a processing unit. And the method further includes operating the processing unit to produce a tube-assembly motion including an axially-reciprocal component so that a first one of the impact surfaces is impacted by the sample as the sample flows in a first axial direction in the tube chamber and a second one of the impact surfaces is impacted by the sample as the sample flows in a second opposite axial direction in the tube chamber during the axially-reciprocal tube-assembly motion to dissociate the sample.

In some embodiments, the method does not include using any enzymes or magnetic forces in the tube chamber. Such methods produce mechanical (and only mechanical) disassociation of the sample in the tube.

In other embodiments, the method includes using mild enzymes in the tube chamber. Such mild enzymes can be for example an enolase, hydroxylase, and/or neutral metalloproteinase enzyme. Such methods produce mechanical disassociation that is enhanced/boosted by mild enzymatic dissociation.

During operation of the processing unit, the impact surfaces facing in directions that are angled from the longitudinal axis of the tube chamber typically results in blunt impact forces on the sample and shearing forces on the sample during dissociation use. Also, the internal thread being helically arranged typically results in a vortical angular flow of the sample with the blunt impact forces on the sample being greater in the first direction that in the second direction, and the shearing forces on the sample being greater in the second direction that in the first direction. Furthermore, the sample typically impacts the internal helical thread multiple times in the first direction and multiple times in the second direction, for example in embodiments in which the internal helical thread defines multiple complete revolutions about the peripheral inner surface of the tube.

In typical embodiments, loading a sample and a buffer includes loading an organically derived sample and a perfusion buffer into a tube assembly, and wherein operating the processing unit includes disassociating the sample into a dissociate of single intact cells. In some such embodiments, loading a sample and a buffer further includes loading a sample and a buffer into a tube assembly so that the sample and the buffer occupy 20 percent to 90 percent of the tube chamber. Also, in some embodiments, operating the processing unit includes operating at a speed of 0.8 to 1.6 m/s, for a time of 30 sec to 60 sec, or both.

The method can include at least one preliminary step before the processing step. The preliminary step can include for example red blood cell (RBC) lysis to eliminate any RBCs from the sample to be processed.

Furthermore, the method can include at least one post-processing step such as further treatments and/or downstream uses of the disassociated sample. The further treatments can include for example subjecting the cells of the disassociated sample to separating, sorting, counting, enriching, purifying, analyzing, and/or other techniques before downstream uses. And the downstream uses can include for example flow cytometry, genomic diagnostics, cell therapies, proteomic diagnostics, cell culturing, cell imaging, NG2 sequencing, extractions, westerns, ADME/Tox, and/or libraries, and some of these further treatments and/or downstream uses can include additional/secondary dissociation processing (e.g., repeating the processing step).

In some embodiments, the disassociated sample is transferred to another tube for the further treatments and/or downstream uses. In other embodiments, the method includes further treatments and/or downstream uses of the disassociated sample while the disassociated sample is still in the same tube. For further treating using a centrifuge, for example, after the processing is complete, the threaded tube assembly containing the dissociated sample is transferred directly to and placed into a centrifuge, and the centrifuge is operated to spin down the dissociated sample to separate out single intact cells from extracellular debris.

Person of ordinary skill in the art will be understood that all details provided herein for any of the tube assembly, the dissociation system, and the dissociation method are applicable to all of the tube assembly, the dissociation system, and the dissociation method. In typical embodiments, the tube assembly can be used as a component of the dissociation system to perform the dissociation method. As such, some of these details are not repeated unnecessarily throughout this disclosure for brevity.

The specific techniques and structures employed to improve over the drawbacks of the prior devices and accomplish the advantages described herein will become apparent from the following detailed description of example embodiments and the appended drawings and claims.

### BRIEF DESCRIPTION OF THE DRAWINGS

FIG. 1 is an exploded view of a processing tube assembly according to an example embodiment, with a tube shown in a cross-section view and having internal helical threads, and with a cap shown in a side view.

FIG. 2 is a top schematic view of the tube of FIG. 1, showing the internal helical threads.

FIG. 3 shows a portion of the tube of FIG. 1, with details of the internal helical threads shown.

FIG. 4 shows the tube of FIG. 1 in processing use according to an example method embodiment, with a circulatory angular flow path of the sample shown as a circulatory directional arrow.

FIG. 5 schematically shows a bottom portion of the tube of FIG. 4 in processing use, with two sequential portions of the sample flow shown as directional arrow segments both in a first axial direction and a first angular direction.

FIG. 6A-6B show a top view of the tube portion of FIG. 5 in processing use, with the two sequential portions of the sample flow shown as two respective angular-direction arrow segments.

FIG. 7 schematically shows the tube portion of FIG. 5 in processing use, with two additional sequential portions of the sample flow shown as directional arrow segments both in an opposite second axial direction and the same first angular direction.

FIGS. 8A-8B show a top view of the tube portion of FIG. 7 in processing use, with the two sequential portions of the sample flow shown as two respective angular-direction arrow segments.

FIG. 9 shows a sequence of operation of the tube of FIG. 4 in processing use, with a plot of tube axial displacement versus time at the top, with the respective axial positions of the sample in the side-view tubes shown in the middle, and with the respective angular positions of the sample in the top-view tubes shown at the bottom.

FIGS. 10-13 are side cross-sectional views of four of the tube assemblies having alternative configurations of the helical thread with different thread pitches.

FIGS. 14, 16, and 18 are top views of three of the tube assemblies having alternative configurations of the helical thread with multiple helix threads, and FIGS. 15, 17, and 19 are respective cross-sectional views taken at lines 15-15, 17-17, and 19-19 of FIGS. 14, 16, and 18.

FIG. 20 includes side cross-sectional views of nine of the tube assemblies having alternative configurations of the helical thread with different thread profiles.

FIG. 21 is a side cross-sectional view of a portion of the tube assembly having an alternative configuration of the helical thread with a recessed helical thread.

FIG. 22 is an exploded perspective view of the tube assembly having an alternative configuration of the helical thread with the helical thread separately formed on an insert sleeve, FIG. 23 is a perspective view of the insert sleeve, and FIG. 24 is a side cross-sectional view of the insert sleeve.

FIGS. 25, 27, 29, and 31 are perspective views of four insert sleeves having alternative configurations with slots, and FIGS. 26, 28, 30, and 32 are respective cross-sectional views of the insert sleeves.

FIGS. 33 and 34 show results of testing done using example method embodiments.

### DETAILED DESCRIPTION OF EXAMPLE EMBODIMENTS

Generally described, the present disclosure relates to laboratory processing devices and methods for dissociating samples into output cells for further downstream uses (e.g., processing, culturing, treating, or analyzing, for life-sciences and other applications). In example embodiments, the processing devices and methods are operable to produce relatively gentle and low-impact disassociation (also referred to as dissolution) of the sample resulting in output cells most of which are preserved as single intact cells suitable for these further uses. In such embodiments, the processing devices and methods can process a wide range of tissue samples in an automated and very consistent manner that results in higher volume and higher throughput and more consistent output. In other embodiments, the processing devices and methods are operable to produce relatively high forces on the sample and thus relatively vigorous, high-impact disassociation or other processing of the sample.

A few preliminary definitions are as follows. These definitions are all in the context of laboratory processing equipment and methods, with the term “laboratory” intended to be broadly construed to mean any type of controlled sterile environment used for processing samples, including clinical diagnostic and pathology laboratories, academic and industry research laboratories, and teaching laboratories.

“Processing” as used herein is intended to be broadly construed to mean high-shear disaggregating, with significant micron-level particle-size reduction (and optionally mixing and/or resuspending) of the components of a sample by an oscillating motion of a tube containing the sample (with the tube sometimes optionally also including another media such as beads). Dissociating is a common but non-exclusive example of such processing.

“Dissociating” as used herein is intended to be broadly construed to mean a type of sample processing that results in separation of tissue into a single cell state. Meaning there are no extra cellular attachments. Cells are free bodies and not sessile. This is not to be confused with anoikis, which is a form of programmed cell death that occurs in anchorage-dependent cells when they detach from the surrounding extracellular matrix. Dissociation is a physical separation, but not destruction, of the cells found within a solid tissue sample, to produce/output a dissociated sample of cells that are intact (i.e., whole, the cell walls are not) and single (i.e., individual and thus not stratified or otherwise joined in some form with any other cells), and typically but not always necessarily viable (e.g., live or otherwise usable for downstream uses) (aka “single intact cells”).

“Processing unit” as used herein is intended to be broadly construed to mean a laboratory device that is operable to drive tubes holding samples through an oscillatory motion including an axially reciprocating component to process the samples. A common but non-exclusive example processing unit is a laboratory homogenizer, for example a bead mill homogenizer such as the BEADRUPTER bead mill homogenizer (Omni International, Inc., Kennesaw, GA), or another conventional bead mill homogenizer or other type of laboratory homogenizer. The processing units produce an oscillatory motion of the tubes holding the samples that can be (a) solely an axially reciprocating motion (e.g., many conventional laboratory homogenizers), (b) a “swashing” motion including an axially reciprocating component and an Abbé offset spinning motion about the axis of reciprocation at the same rate as the oscillation (e.g., the BEADRUPTER bead mill homogenizer), or (c) any other oscillatory motion as long as it includes an axially reciprocating component.

“Sample” as used herein is intended to be broadly construed to include any type of organically derived material (i.e., organic matter and/or derived from organic matter) that can be processed and for which dissociation or other processing could be useful in life-science applications, such as but not limited to human and/or non-human bodily fluid and/or tissue (e.g., blood, bone-marrow cells, a coronary artery segment, or a piece of an organ), other organic matter (e.g., plants or food), and/or other chemicals or substances.

“Tube” is intended to be broadly construed to include any closable/sealable vessel or container that can hold a sample during processing and is not limited to conventional cylindrical hard-plastic vials with screw-on caps and thus also includes well plates, polygonal or other-shaped vials, and other types of vessels and/or closures.

And “downstream uses” is intended to be broadly construed to include processing, culturing, treating, and/or analysis of single intact cells for life-sciences and other applications. A non-exclusive list of common downstream uses includes extractions, westerns, ADME/tox, flow cytometry, genomic diagnostics, cell therapy, proteomic diagnostics, cell cultures, libraries, cell imaging, and NG2 sequencing.

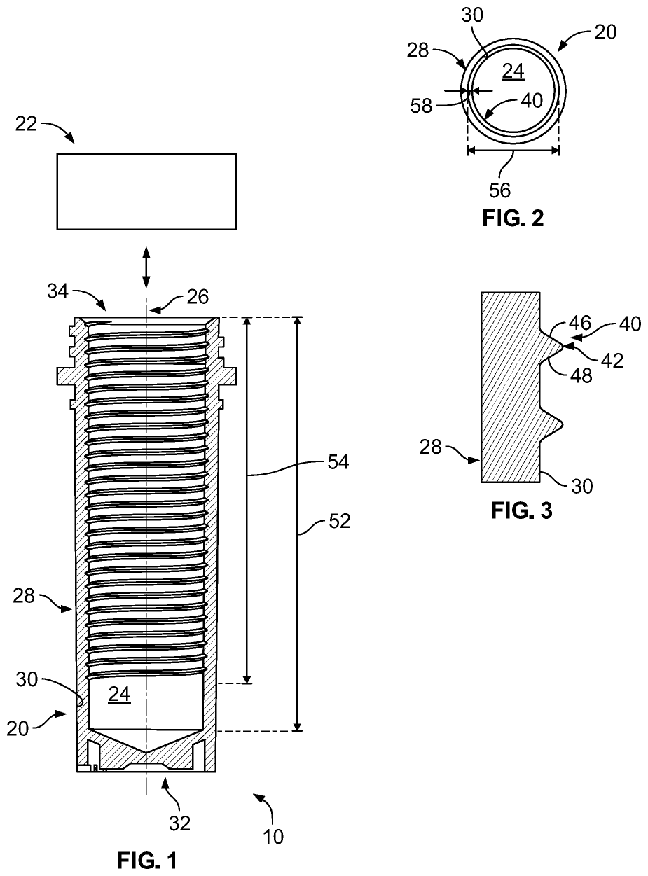

FIGS. 1-9 show a processing tube assembly 10 according to an example embodiment. The tube assembly 10 can be used for dissociation of a sample 2, for example to produce output cells most of which are single and intact cells. This is done using a processing unit (not shown) that can be operated to generate an oscillating motion with an axially reciprocating component. The processing unit that the tube assembly 10 can be used with to produce the intended single intact cells can be of a conventional type such as that defined herein.

It will be noted that current protocols do not differentiate between recovered cells (all single intact cells, whether viable or not) and the total count (additionally including debris from damaged cells), and so there is no benchmark for measurements. In some embodiments, the majority of the output is not single intact cells, but in any event a significant portion of the output is single intact cells relative to conventional methods and equipment. For example, in some preliminary tests only about 10 percent (e.g., about 10 percent to about 15 percent) of the output was single intact cells, based on the total count. Nevertheless, in most embodiments, when the output is defined as recovered cells (excluding debris from damaged cells), the majority of the output cells are single and intact.

The tube assembly 10 includes a processing tube 20 and a cap 22 that removably couple together to form a tube interior chamber 24 that has a longitudinal axis 26 and that contains the sample 2 during dissociation. Except as noted herein, the processing tube 20 and the cap 22 can be of a conventional type that are commercially available in the laboratory sample-processing industry. For example, the tube 20 can include a peripheral (e.g., cylindrical) sidewall 28 defining a peripheral (e.g., cylindrical) inner surface 30 peripherally bounding the interior chamber 24 (with the tube longitudinal axis 26 parallel to the tube inner surface 30), a bottom wall 32 defining and bounding (closing off) the bottom of the interior chamber 24, and an open top 34 for adding and removing the sample 2 when the cap 22 is removed. Also, the cap 22 can be for example a screw-top design, with internal threads that mate with external threads on an upper portion of the tube sidewall 24. Example tubes 20 have an internal chamber 24 with a volume of 2 mL, 7 mL, or another standard or specialized size as may be desired for a given application (e.g., a 7 mL or other relatively larger tube generally works better for samples that require a relatively larger physical sample size such as lung or liver samples, and a 2 mL or other relatively smaller tube generally works better for samples that do not require a relatively larger physical sample size such as spleen samples).

In example embodiments, the tube assembly 10 holding the sample 2 does not also hold grinding media (e.g., beads or active grinding media) during processing use. In other embodiments, the tube assembly 10 holds the sample 2 and also grinding media during processing.

To provide for the desired sample dissociation, the tube 20 includes at least one internal thread 40 extending inwardly from the inner surface 30 of the peripheral sidewall 28 and into the interior chamber 24. The internal thread 40 extends peripherally about (e.g., around) the peripheral inner surface 30 of the tube 20 in a helical arrangement, that is, in a screw-thread arrangement. The internal helical thread 40 includes a ridge 42 defining two impact surfaces 46 and 48 that face in generally opposite directions and are angled with respect to each other. Also, the two impact surfaces 46 and 48 are each at a non-perpendicular angle relative to the inner surface 30 of the tube 20 (see FIG. 3) and relative to the longitudinal axis 26 of the tube chamber 24 (the longitudinal axis 26 is parallel to and coaxial with the inner surface 28).

The impact surfaces 46 and 48 of the helical thread 40 thereby form angled surface areas that are configured/arranged (e.g., positioned and oriented) to, during processing use (when subjected to the axially-reciprocal motion), be impacted by the sample 2 to induce a swirling flow path 50 of the sample 2 that forms a vortex within the tube chamber 24. In this way, the vortical angular flow 50 of the sample 2 causes it to swirl about and impact and the internal thread 40 and the tube sidewall inner surface 30 (as well as already dissociated sample particles after startup) to dissociate the sample 2 into the output cells.

Furthermore, the vortical flow 50 of the sample 2 results in a longer travel distance of the sample 2 within the tube chamber 24 during each oscillation (relative to a purely axial/linear reciprocating flow), and thus more impacts of the sample 2 against the tube sidewall inner surface 30, than for conventional processing tubes. And the inclusion of the internal thread 40, which induces the vortical flow 50 of the sample 2, results in additional impacts of the sample 2. These additional impacts of the sample 2 (against the impact surfaces 46 and 48 of the internal peripheral thread 40 and against the tube inner surface 30) during processing use cause the sample 2 to separate into a dissociate of single intact cells at oscillatory speeds low enough to avoid cell damage.

The motion of the tube 20 imparted by the processing unit creates the swirling flow path 50. In example embodiments, the orientation of the helical thread 40 is such that impacts from the thread 40 on the sample 2 are enhanced and/or optimized. It should be noted that the tube 20 imparts energy to the sample 2, so technically the sample 2 is not impacting the helical thread 40, but the helical thread 40 is impacting the sample 2, and the impacts described herein are intended to refer to impacts due to relative movement between the helical thread 40 and the sample 2. It should be further noted that while the swirling vortical flow 50 results from the tube 20 having the helical thread 40, the force and effect of the swirling vortical flow 50 can be enhanced by using a processing unit that imparts a swashing motion to the tube 20.

In particular, the vortical flow 50 of the sample 2 results in a longer travel distance of the sample 2 within the tube chamber 24 during each oscillation, and thus more impacts of the sample 2 against the tube sidewall inner surface 30, than for conventional processing tubes. And the inclusion of the internal thread 40, which induces the vortical flow of the sample 2, results in additional impacts of the sample 2 against the impact surfaces 46 and 48 of the internal thread 40. The result is that, in example embodiments, the vortical angular flow of the sample 2 impacting against the impact surfaces 46 and 48 of the internal peripheral thread 40 and against the tube inner surface 30 during processing use causes the sample 2 to separate into a dissociate of single intact cells, without the need to use enzymes or magnetic forces in the tube chamber 24.

In some embodiments, the internal helical thread 40 extends along only a portion of the entire length 52 of the inner surface 30 of the tube 20. The portion of the inner surface having the helical thread 40 can be an upper portion (adjacent the open top 34), a lower portion (adjacent the bottom wall 32), a middle portion (between the upper and lower portions), or a combination of these. For example, the internal helical thread 40 can extend along only a partial length 54 of the entire length 52 of the inner surface 30 of the tube 20, with the partial length 52 including the upper and middle portions of the inner surface 30, and with the thread 40 not extending to the lower portion of the inner surface 30, as depicted. In testing, good results were obtained for embodiments in which the partial length 54 was over about 50%, for example about 80 percent to about 90 percent (e.g., about 90 percent) of the entire length 52 of the inner surface 30. In other embodiments, the internal thread extends along the entire length of the inner surface 30 of the tube 20.

Also, the ridge 42 of the internal helical thread 40 typically extends inwardly only a relatively small radial distance into the tube chamber 24, with the diameter 56 of the tube chamber 24 being significantly larger than the height 58 of the thread ridge 42. The thread height (also depth) 58 is selected to be large enough to induce the vortical flow 50 of the sample 2 and to provide increased surface area for sample impacts, but not so larger to significantly throttle or disrupt the sample flow. The thread height 58 is typically at least about 5 percent, for example about 10 percent to about 30 percent (e.g., about 14 percent), of the tube diameter 56. In other embodiments, the thread height is relatively larger or smaller relative to the tube radius.

The thread pitch of the internal helical thread 40 is typically about 3 threads per inch to about 32 threads per inch, for example about 15 threads per inch to about 25 threads per inch (e.g., about 20 threads per inch). In addition, the profile of the helical thread 40 typically has a thread angle of about 40 degrees to about 90 degrees, for example about 25 degrees to about 75 degrees (e.g., about 60 degrees) so the oppositely facing impact surfaces 46 and 48 are angled relative to each other and relative to the inner surface 30. In other embodiments, the helical thread has a different thread pitch and/or thread angle.

Also, the internal helical thread 40 of some embodiments forms a single continuous helical ridge 42, for example as depicted. In addition, the helical thread 40 typically defines multiple (e.g., at least two) complete revolutions about the inner surface 30 of the tube 20. In other embodiments, the helical thread includes a plurality of ridge segments in a helical arrangement, defines less than two complete revolutions, or a combination thereof.

In typical embodiments, the internal helical thread 40 is integrally formed with the tube 20 as a single piece (e.g., as depicted). Also, the internal helical thread 40 is typically made of a rigid material, which can be the same hard plastic or other material as the tube 20 is made of (e.g., as depicted). Furthermore, the internal helical thread 40 is typically fixed in place relative to the tube 20 and during use does not move relative to another thread (mating or not) or other element of the tube 20 (e.g., as depicted). In other embodiments, the helical thread is formed on a separate piece that inserts into a standard conventional tube (e.g., as described below) and/or can be made of a softer material with relatively more resiliency to provide a spring effect that accelerates the sample 2 being processed in the tube.

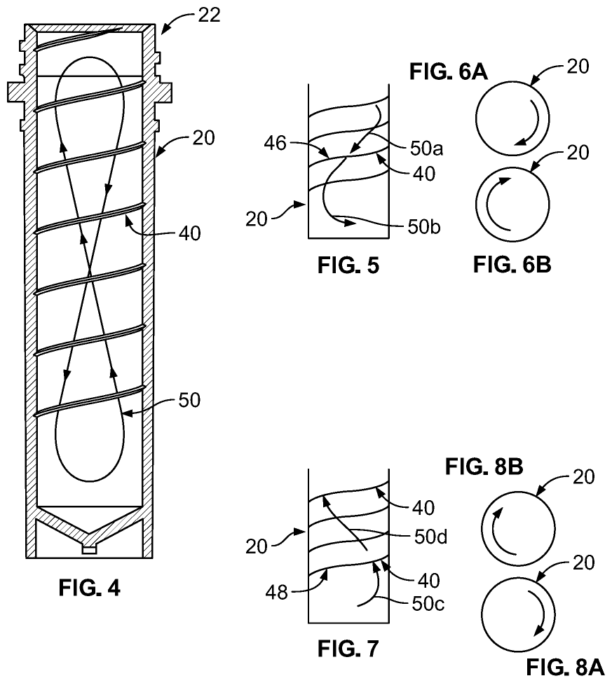

Referring particularly to FIGS. 4-9, a method of using the tube assembly 10 to dissociate a sample 2 will now be described. The method can be performed using the tube assembly 10 of FIGS. 1-3 or any other of the tube assemblies described herein, with the method of dissociating a sample 2 being another example embodiment.

The method includes loading the sample 2 and a buffer into a tube assembly 10. The buffer can include for example chemical reagents such as a liquid perfusion buffer material of a conventional type. In typical embodiments, the sample 2 and the buffer occupy about 10 percent to about 90 percent, for example about 10 percent to about 30 percent (e.g., about 20 percent), of the tube chamber 24. Thus, for a tube assembly 10 having a tube chamber 24 with a volume of about 2 mL, the volume of the sample 2 and the buffer loaded into it can be about 200 uL to about 1800 uL to meet the about 10 percent to about 90 percent range, about 200 uL to about 600 uL to meet the about 10 percent to about 30 percent range about 400 uL to meet the about 20 percent value. Generally, a relatively low volume of the sample 2 and buffer provides enough head space (for the intended disassociation), but not too much head space (for keeping the yield of output cells high). After the sample 2 and buffer are loaded into the tube 20, the tube is sealed (e.g., by the cap 22).

Then the sample-loaded tube assembly 10 is mounted to a processing unit. The processing unit can be of a conventional type such as that defined herein.

Then the processing unit is operated to produce an oscillating tube-assembly motion that includes an axially-reciprocal component. Because of the angular flow induced by the helical thread 40, the oscillatory reciprocating motion of the tube assembly 10 can be at a relatively lower speed than is conventionally used for processing applications. The speed is selected for causing the sample 2 to flow along/across the helical thread 40 of the tube 20 and not be totally disrupted by other internal forces. (It is believed that as some single intact cells become disassociated, they tend to collect and “hide” within the recesses between the ridges of the helical thread 40, which provide some protection from damage, when using relatively lower processing speeds, whereas using relatively higher speeds would tend to pull more of the single intact cells from these cavities and damage or destroy them.

For example, the processing unit can be operated at a speed of about 0.8 m/s to about 8.0 m/s, for example from about 0.8 m/s to about 2.4 m/s, to about 2.8 m/s, or to about 3.2 m/s (e.g., about 1.6 m/s), and/or for a time of from about 30 sec to about 150 sec, for example about 30 sec to about 60 sec (e.g., about 45 sec), to disassociate the sample into a dissociate/output of single intact cells. The operating time can be in cycles, for example, one cycle of 60 sec, or four cycles of 15 sec each. These operating speeds and times are typically suitable for an oscillatory stroke length of for example about 0.8 inches to about 3.0 inches displacement, for example about 1.2 inches. It will be understood that the processing unit can be operated at other speeds and for other times, with other oscillatory stroke lengths, to produce the intended output dissociate of single intact cells.

During processing use, with the tube assembly 10 driven through the oscillating motion including the axially-reciprocal component, the helical thread 40 induces a circulatory flow 50 of the sample 2 in the tube chamber 24, as shown in FIG. 4. So the first impact surfaces 46 are impacted by the sample 2 when the sample flow 50 is in a first axial direction in the tube chamber 24, as shown in FIGS. 5 and 6A. then the processing unit operates to reverse the axial direction of the oscillatory motion, and thus the sample flow 50 reversed, as shown by FIGS. 5 and 6B and FIGS. 7 and 8A. Then the second impact surfaces 48 are impacted by the sample 2 as the sample flow 50 is in a second opposite axial direction in the tube chamber 24, as shown in FIGS. 7 and 8B.

In this way, the helical thread 40 induces the circulatory angular flow 50 of the sample 2 (e.g., in the form of a vortex), with the angular direction of the vortical flow 50 the same in both axial directions, as shown in FIGS. 5 and 6A-B and FIGS. 7 and 8A-B. Thus, when the sample 2 moves in the first axial direction in the tube 20, it has a relatively high angle of attack on the helical thread 40 (the angular flow 50 more directly opposes the helical thread 40 of the tube 20 in this stage of the oscillatory stroke), and when the sample moves in the second opposite axial direction it has a relatively low angle of attack. Typically, the first axial direction is the same direction as the initial stroke in the oscillatory motion, because during that initial stroke the helical thread 40 will set up a vortical flow 50 in an angular direction (with the vortical flow enhanced when using a processing unit imparting a swashing motion to the tube). And even though the helical thread 40 is counter to the angular direction of the vortical flow 50 in the second axial direction, the helical thread 40 has a sufficiently small height (and thus sample-contacted surface area) that it does not impart enough force on the sample 2 to reverse the angular flow direction in the second axial direction.

In addition, because the impact surfaces 46 and 48 face in directions that are angled from the longitudinal axis of the tube chamber 24, they impart blunt impact forces on the sample 2 and also shearing forces on the sample 2 during dissociation processing use. The blunt impact forces on the sample 2 are greater in the first direction that in the second direction, and the shearing forces on the sample are greater in the second direction that in the first direction, because the vortical sample flow 50 is in the same angular direction during both axial directions of the oscillatory motion. This is because the sample flow 50 impacts the first impact surface 46 at a first angle in the first direction (see FIGS. 5 and 6A) and impacts the second impact surface 48 at a second angle in the second direction (see FIGS. 7 and 8A), with the second impact angle being closer to perpendicular than the first impact angle. In some embodiments, the second impact angle is perpendicular, while the first impact angle is always non-perpendicular.

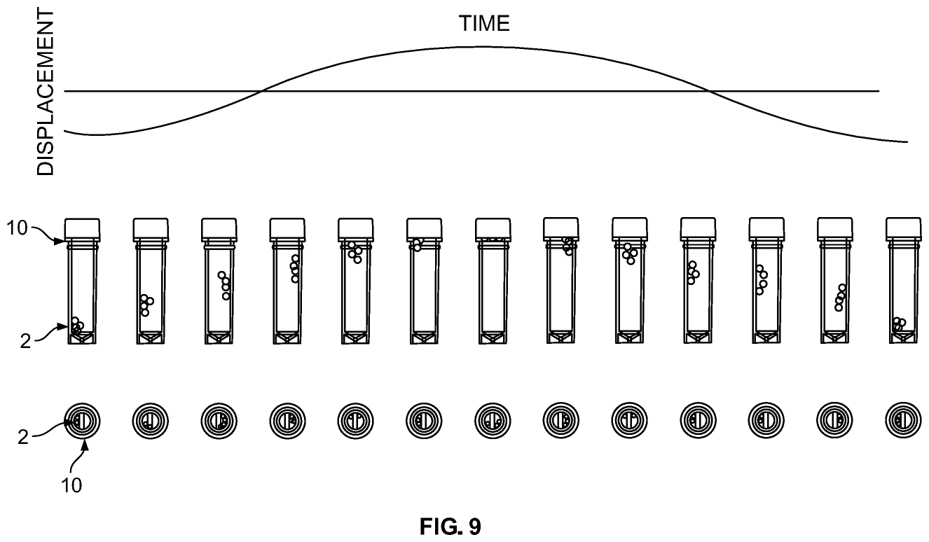

FIG. 9 further shows this angular flow 50 of the sample 2 within the threaded tube assembly 10 (the thread is not shown for simplicity). This figure includes a plot of tube axial displacement (the oscillatory reciprocating motion) versus time at the top. Below that, there are a series of tube assemblies shown, with each of the depicted tube assemblies including a sample 2 in a respective axial position corresponding to the axial displacement immediately above it. Below that, there are a series of tube assemblies shown, with each of the depicted tube assemblies including a sample 2 in a respective angular position corresponding to the axial position immediately above it. Collectively, this figure shows the circulatory angular sample flow over one oscillation of the tube assembly 10

The result of the oscillatory reciprocating motion of the helically threaded tube assembly is that the sample 2 is dissociated into an output dissociate of single intact cells. Furthermore, this method can accomplish this result without using enzymes, magnetic forces, or moving mechanical parts in the tube assembly 10. As such, the method produces purely mechanical dissociation.

It should be noted that the methods typically include at least one preliminary step before the processing step. The preliminary step can include for example red blood cell (RBC) lysis to eliminate any RBCs from the sample to be processed. Such preliminary steps are typically performed before the sample is inserted into the tube to be used in the professing step, though in some embodiments the preliminary step can be performed with the sample in the sane tube to be used in the processing step.

Other method embodiments are the same except they are performed with the tube including an enzyme in addition to the sample, and as such they product enzymatically assisted/boosted mechanical dissociation. The enzyme can be included in a buffer used in the method. The enzyme can be a mild (soft) type (class) of enzyme (i.e., milder (softer/less aggressive) relative to the aggressive enzymes conventionally used in enzymatic dissociation such as serine proteases and hydrolases), that is, the enzyme is selected so that in the presence of the sample it produces a biochemical reaction that does not break down and/or destroy the cell walls of the sample and thus causes no more than negligible transcriptome aberrations while yielding single intact cells. For example, the enzyme can be an enolase, hydroxylase, and neutral metalloproteinase (e.g., collagenase and anion enzymes) enzyme, or another enzyme that does not break down and/or destroy the cell walls of the sample during use in this mechanical dissociation method. In such methods, the samples are incubated in a formulation of mild enzymes selected to help loosen single cells from within sample tissues. The mild enzymes cut the triple helical protein chains of collagen, which are found in the extracellular matrix in all mammalian collagen structures, resulting in cell isolation.

The methods disclosed herein all include mechanically (and optionally additionally enzymatically) disassociating the sample (into an output dissociate of single intact cells) using a threaded tube assembly and a processing unit (for example as described herein) into single intact cells. Another example embodiment is a method of intra-tube processing of a sample according to any of the processing methods disclosed herein, and including post-processing steps such as further treatments and/or downstream uses of the disassociated sample. (In some methods, the single intact cells are directly used in downstream uses, and in others they are further treated before the downstream uses).

Typically, the output dissociated sample is a heterogenous cell population including primarily the desired single intact cells and also some debris (e.g., cells that are non-viable (dead and/or damaged) and thus not suitable for downstream uses, connective tissue, etc.). Thus, the further treatments can include subjecting the cells of the disassociated sample to separating, sorting, counting, enriching, purifying, analyzing, and/or other techniques before downstream uses. For example, the further treatments can include using a magnetic cell separation system (e.g., the MOJO SORT system by BioLegend, Inc.), a conventional laboratory centrifuge, or another conventional or new system that isolates, purifies, and sorts the single intact cells in the heterogenous cell population of the dissociated sample. The further treatments can also include using a dual-fluorescence cell counter (e.g., the CELLOMETER K2 system by Nexcelom Bioscience LLC), a high-performance integrated virtual environment (HIVE) sequencing system, or another conventional or new system that counts and optionally analyses the single intact cells in the heterogenous cell population of the dissociated sample. And the downstream uses can be of the type described herein, including flow cytometry, genomic diagnostics, cell therapies, proteomic diagnostics, cell culturing, cell imaging, NG2 sequencing, extractions, westerns, ADME/Tox, and/or libraries. Some of these downstream-use steps can include further processing using the mechanical dissociation methods disclosed herein (i.e., additional/secondary processing of the dissociated sample, in the same or another threaded tube assembly, using the same or another processing unit, with or without any added steps/features such as filtration).

In some embodiments, the disassociated sample is transferred to another tube for the further treatments and/or downstream uses. In other embodiments, the method includes further treatments and/or downstream uses of the disassociated sample while the disassociated sample is still in the same tube (which improves efficiency/yields by minimizing single intact cells wasted/lost in the tube transfer). For such further treating, after the processing is complete, the threaded tube assembly containing the dissociated sample is transferred directly to and placed into the further treatment system/device (e.g. centrifuge, MOJO SORT device, CELLOMETER K2 device, etc.). The dissociated sample does not need to be removed from the threaded tube assembly it was processed in and transferred to a separate tube assembly before placing the dissociated sample in the further treatment system/device.

For further treating using a centrifuge, for example, after the processing is complete, the threaded tube assembly containing the dissociated sample is transferred directly to and placed into a centrifuge.

Next the centrifuge is operated to spin down the dissociated sample to separate out the single intact cells. These single intact cells are still in the tube chamber the sample was loaded into before processing, now separated as supernatant with the debris pushed down. This is important to remove the debris from the single intact cells of the dissociated sample. Then the single intact cells can be removed from the threaded tube assembly (the same one the sample was initially loaded into). These single intact cells can then be further treated and/or used in downstream uses for various life sciences applications.

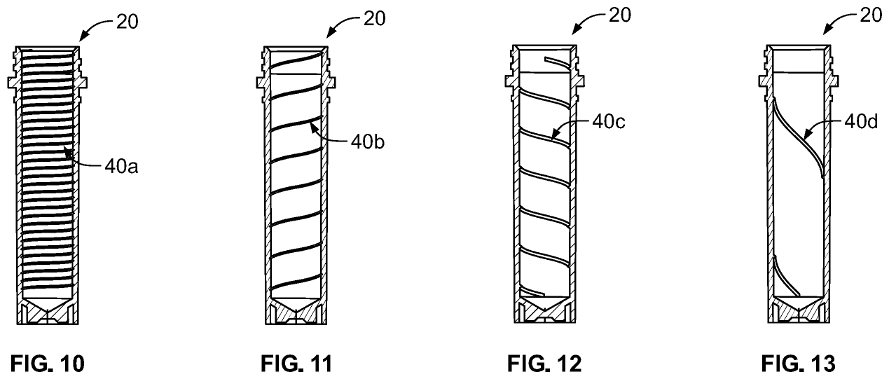

The tube assembly 10 can be provided with helical threads in a variety of alternative configurations. FIGS. 10-32 shown just some of the alternative configurations of the helical thread. It will be understood that the different aspects of these different configurations can be combined into additional example embodiments, with these additional example embodiments not shown and described for brevity.

For example, FIGS. 10-13 show the tube 20 having alternative configurations (thread pitches) of the helical thread. In FIG. 10, the helical thread 40a has the same high thread pitch as the threaded tube assembly 10 of FIGS. 1-9, except the thread 40a extends along the entire length of the tube. In FIG. 11 the helical thread 40b has a medium-high thread pitch, in FIG. 12 the helical thread 40c has a medium-low thread pitch, and in FIG. 13 the helical thread 40d has a low thread pitch. In other embodiments, the helical thread has other thread pitches. Also, the helical thread is a right-handed helix FIGS. 12-13, whereas it is left-handed in other figures (the handedness of the helix can be selected for enhanced impacts based on the motion produced by the processing unit).

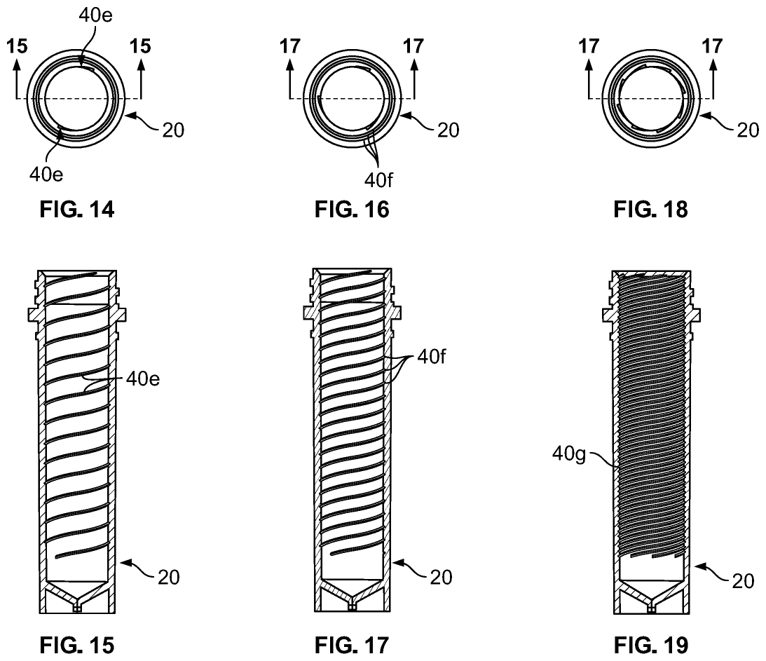

In addition, FIGS. 14-19 show the tube 20 having alternative configurations (multiple helices) of the helical thread. In FIGS. 14-15 the tube 20 has double helical threads 40e (i.e., two helical thread ridges in a double helix arrangement), in FIGS. 16-17 it has triple helical threads 40f, and in FIGS. 18-19 it has eight-start helical threads 40g. The helical threads in these figures have multiple starts or multiple ridges/groves that are intertwined to increase the number of active features while maintaining suitable threads per inch. In other embodiments, the helical thread has other helical arrangements.

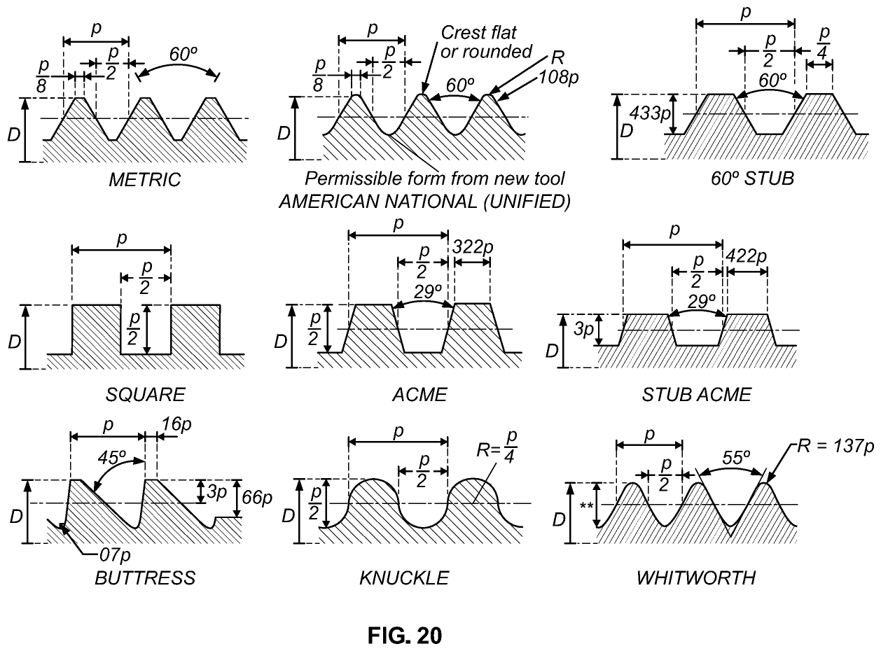

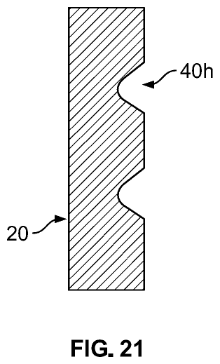

Furthermore, FIG. 20 shows the tube 20 having nine alternative configurations (profiles) of the helical thread that can be selected and used to adjust how aggressive the disassociation is. These can include metric, American national (unified), 60 degree stub, square, ACME, stub ACME, buttress, knuckle, and Whitworth. For an internal helical thread with a square profile, the thread angle is zero degrees, so the oppositely facing impact surfaces are parallel to each other. In other embodiments, the helical thread has other profiles, for example with serrations, castellations/notches, or undulations. In FIG. 20, D is the thread height, P is the thread pitch, and the dimensions shown are representative (non-limiting) examples. Also, FIG. 21 shows the tube 20 having an alternative configuration with a recessed helical thread 40h.

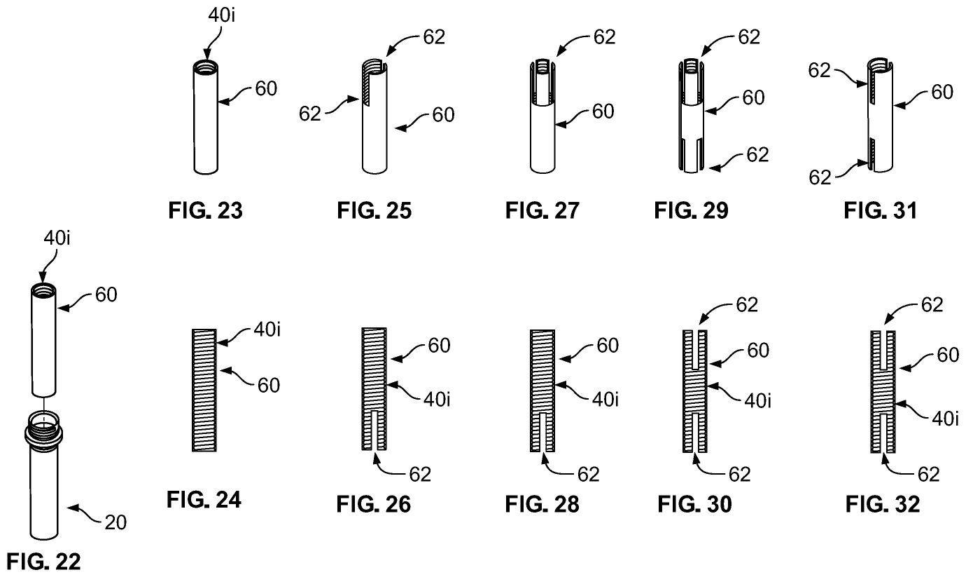

Moreover, FIGS. 22-32 show the tube 20 having alternative configurations (separately formed) of the helical thread. In these embodiments, the internal helical thread is formed on a sleeve 60 that inserts into the tube 20 and forms the internal peripheral surface of the tube. In FIGS. 22-24 the insert sleeve 60 has the same helical thread 40i as in the embodiment of FIG. 10. In FIGS. 25-26 the insert sleeve 60 has the same helical thread 40i and also includes two top slots 62, in FIGS. 27-28 the insert sleeve 60 has the same helical thread 40i and also includes four top slots 62, in FIGS. 29-30 the insert sleeve 60 has the same helical thread 40i and also includes four top slots 62 and four bottom slots 62, and in FIGS. 31-32 the insert sleeve 60 has the same helical thread 40i and also includes two top slots 62 and two bottom slots 62. The top slots 62 can be provided to stop rotation or facilitate draft in the tube 20 to make a tight fit, and as such they are optional. In other embodiments, the insert sleeve has slots in other configurations.

As described above, example embodiments include processing tube assemblies including internal helical threads for use with separately provided processing units, as well as methods of dissociating samples using such tube assemblies with such processing units. Additional embodiments include processing systems including such tube assemblies and such processing units, for example bead mill homogenizers that produce a swashing tube-assembly motion including the axially-reciprocal component.

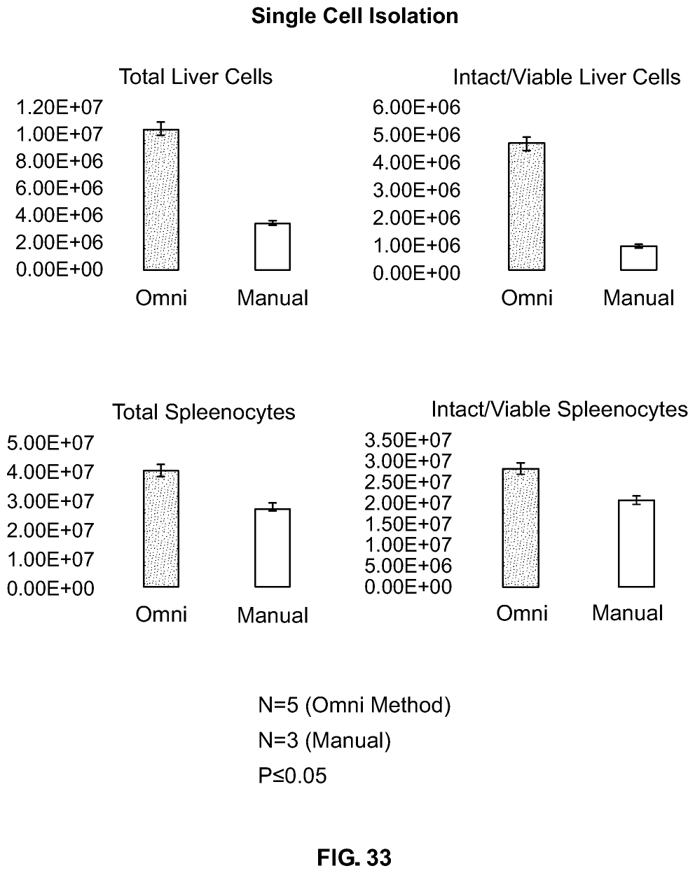

Testing was done to demonstrate effectiveness and identify processing parameters of a selected embodiment (labeled “Omni”) compared to traditional manual mechanical dissection/dissociation (labeled “Manual”), and the test results are shown in FIG. 33. The testing was done using an intact left lateral liver lobe (top two graphs) and an entire spleen (bottom two graphs) obtained from fresh (not frozen or fixed) murine tissue samples. The reported/shown test results are averages of a total number (N) of five Omni tests and three manual tests, with a standard deviation/variance (P) of less than 0.05. All cell counts were recorded and shown in the figure as cells/mL of the sample.

The selected embodiment used in the Omni testing included the BEADRUPTOR ELITE bead mill homogenizer (Omni International, Inc.) as the professing unit and the L-20 2 mL processing tubes (Omni International, Inc.) adapted to include internal helical threads as shown in FIGS. 1-9 and described with respect to those figures. In particular, the processing tubes used in the Omni testing included an internal helical thread forming a continuous helical ridge extending along the entire length of the internal chamber of the tube, with the helical ridge made of the same polymer material as the tube, and with the helical ridge having 20 threads per inch and a 60-degree thread angle. The homogenizer processing unit was operated at a 1.6 m/s speed for 35 secs.

Also, the processing tubes in the Omni testing contained the samples and a PBS buffer including a collagenase IV and DNASE I enzyme. The samples were not perfused.

On the other hand, the dissection/disassociation in the manual testing was performed in a conventional manner by vigorously and forcefully hand-stirring tweezers to disassociate the samples in petri dishes for 20 minutes, by hand (by a graduate student, with the physical capabilities of an average adult human, as fast and forcefully as could be reasonably sustained), without using a powered device. An enzyme was not used, a buffer was not used, and the samples were not perfused.

As can be seen in FIG. 33, the Omni method produces more total cells than the manual method, as shown in the top left graph for liver cells and the bottom left graph for spleen cells. As further shown, of those total cells produced, the Omni method produces more single intact (labeled intact/viable) cells than the manual method, as shown in the top right graph for liver cells and the bottom right graph for spleen cells. These test results demonstrate that the Omni method provides a significantly higher throughput than the manual method, producing many more single intact cells in much less time.

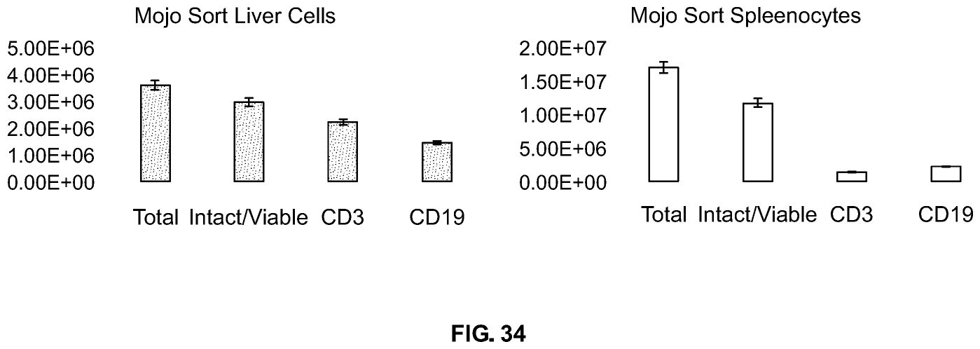

These single intact cells are now available for downstream uses (e.g., tissue culture or analysis, or other downstream uses as disclosed herein), or for further treating before such downstream uses. As noted above, the further treating can include cell sorting, for example using the MOJO SORT system. Additional testing was done to show the effectiveness of the Omni processing method in combination with a post-processing sorting step. The test results are shown in FIG. 34, with the left graphs showing post-processing results for the liver cells produced by the Omni method of FIG. 33 and the right graphs showing the post-processing results for the spleen cells produced by the Omni method of FIG. 33. The sorting step was performed using the MOJO SORT system, specifically the MOJO SORT Human CD4 Nanobeads (#480013) and the MOJO SORT Human CD19 Selection Kit (#480105).

As can be seen in FIG. 34, the post-processing sorting step confirms that Omni method produces a large number of total cells with a high percentage of them being single intact (labeled intact/viable) cells. In addition, the test results show that relatively high percentage of CD3 and CD19 (T and B cells), which verifies that the test results are accurate.

There is always the possibility of variability between biological samples from different areas of a given tissue and variance between different organisms. Gross anatomical evaluation was conducted of each animal to rule out of overt abdominal pathology. However, without histopathology and molecular analysis of all tissues utilized in these experiments, we are unable to guarantee the absence of pathology in any of the animals utilized for these experiments. All samples from murine models were prepared in the same method by the same scientist for each of the included experiments in an attempt to reduce variation in sample preparation before tissue dissociation experiments.

It is to be understood that this disclosure is not limited to the specific devices, methods, conditions, or parameters of the example embodiments described and/or shown herein, and that the terminology used herein is for the purpose of describing particular embodiments by way of example only. Thus, the terminology is intended to be broadly construed and is not intended to be unnecessarily limiting of the claimed subject matter. For example, as used in the specification including the appended claims, the singular forms “a,” “an,” and “the” include the plural, the term “or” means “and/or,” and reference to a particular numerical value includes at least that particular value, unless the context clearly dictates otherwise. Also, any use of the terms “about,” “substantially,” and/or “generally” are intended to mean the exact value or characteristic indicated, as well as close approximations that are understood by persons of ordinary skill in the art to be sufficiently close to the exact value or characteristic based on the context of the intended use and application. In addition, any methods described herein are not intended to be limited to the sequence of steps described but can be carried out in other sequences, unless expressly stated otherwise herein.

While the claimed embodiments have been shown and described in example forms, it will be apparent to those skilled in the art that many modifications, additions, and deletions can be made therein without departing from the spirit and scope of the disclosure as defined by the following claims.

What is claimed is:

1. A laboratory processing tube assembly that mounts to a processing unit for dissociating an organically derived sample by an oscillating tube-assembly motion including an axially-reciprocal component, the tube assembly comprising:
a tube and a cap that removably couple together to form a tube chamber that has a longitudinal axis and that contains the sample during dissociation, wherein the tube has a peripheral inner surface; and
at least one internal thread extending inwardly from and peripherally about the inner surface of the tube in a helical arrangement, wherein the internal helical thread includes a ridge defining two impact surfaces facing in generally opposite directions at a non-perpendicular angle relative to the longitudinal axis of the tube chamber and relative to the inner surface of the tube, and wherein a first one of the impact surfaces is impacted by the sample as the sample flows in a first axial direction in the tube chamber and a second one of the impact surfaces is impacted by the sample as the sample flows in a second opposite axial direction in the tube chamber during the axially-reciprocal tube-assembly motion to dissociate the sample.

2. The laboratory processing tube assembly of claim 1, wherein the sample impacts against the impact surfaces of the internal helical thread during dissociation use and separates the sample into a dissociate of single intact cells, without using any enzymes or magnetic forces in the tube chamber, or with using a mild enzyme in the tube chamber.

3. The laboratory processing tube assembly of claim 1, wherein the impact surfaces facing in directions that are angled from the longitudinal axis of the tube chamber results in blunt impact forces on the sample and shearing forces on the sample during dissociation use.

4. The laboratory processing tube assembly of claim 3, wherein the internal helical thread induces angular flow of the sample, wherein the blunt impact forces imparted by the internal helical thread on the angularly flowing sample are greater in the first direction that in the second direction, and wherein the shearing forces imparted by the internal helical thread on the sample are greater in the second direction that in the first direction.

5. The laboratory processing tube assembly of claim 1, wherein the internal helical thread forms a single continuous ridge.

6. The laboratory processing tube assembly of claim 1, wherein the internal helical thread extends along 80 percent to 90 percent of an entire length of the internal peripheral surface of the tube.

7. The laboratory processing tube assembly of claim 1, wherein the internal helical thread defines at least two complete revolutions about the peripheral inner surface of the tube.

8. The laboratory processing tube assembly of claim 1, wherein the internal helical thread has a thread pitch of 15 threads per inch to 25 threads per inch.

9. The laboratory processing tube assembly of claim 1, wherein the internal helical thread has a square profile with a zero degree thread angle so the oppositely facing impact surfaces are parallel to each other.

10. The laboratory processing tube assembly of claim 1, wherein the internal helical thread has a profile with a thread angle of 25 degrees to 75 degrees so the oppositely facing impact surfaces are angled relative to each other.

11. The laboratory processing tube assembly of claim 1, wherein the internal helical thread is formed on a sleeve that inserts into the tube and forms the peripheral inner surface of the tube.

12. The laboratory processing tube assembly of claim 1, wherein the internal helical thread is formed by two ridges in a double helix arrangement.

13. A laboratory dissociation system for dissociating a sample, comprising:
a processing unit that produces an oscillating tube-assembly motion including an axially-reciprocal component; and
a tube assembly that mounts to the processing unit, wherein the tube assembly includes a tube and a cap that removably couple together to form a tube chamber that has a longitudinal axis and that contains the sample during dissociation use, wherein the tube has a peripheral inner surface and at least one internal thread extending inwardly from and peripherally about the peripheral inner surface in a helical arrangement, wherein the internal helical thread includes a ridge defining two impact surfaces facing in generally opposite directions at a non-perpendicular angle relative to the longitudinal axis of the tube chamber and relative to the inner surface of the tube, and wherein a first one of the impact surfaces is impacted by the sample as the sample flows in a first axial direction in the tube chamber and a second one of the impact surfaces is impacted by the sample as the sample flows in a second opposite axial direction in the tube chamber during the axially-reciprocal tube-assembly motion to dissociate the sample.

14. The laboratory dissociation system of claim 13, wherein the laboratory processing unit is a bead mill homogenizer.

15. The laboratory dissociation system of claim 14, wherein the tube-assembly motion produced by the bead mill homogenizer is a swashing motion including the axially-reciprocal component.

16. A laboratory sample dissociation method, comprising:
loading a sample and a liquid buffer into a tube assembly including a tube and a cap that removably couple together to form a tube chamber that has a longitudinal axis and that contains the sample during dissociation use, wherein the tube has a peripheral inner surface and at least one internal thread extending inwardly from and peripherally about the inner surface in a helical arrangement, wherein the internal helical thread includes a ridge defining two impact surfaces facing in generally opposite directions at a non-perpendicular angle relative to the longitudinal axis of the tube chamber and relative to the inner surface of the tube;
mounting the loaded tube assembly to a processing unit; and
operating the processing unit to produce a tube-assembly motion including an axially-reciprocal component so that a first one of the impact surfaces is impacted by the sample as the sample flows in a first axial direction in the tube chamber and a second one of the impact surfaces is impacted by the sample as the sample flows in a second opposite axial direction in the tube chamber during the axially-reciprocal tube-assembly motion to dissociate the sample, without using any enzymes or magnetic forces in the tube chamber, or with using a mild enzyme in the tube chamber.

17. The laboratory sample dissociation method of claim 16, wherein loading a sample and a buffer includes loading an organically derived sample and a perfusion buffer into a tube assembly, and wherein operating the processing unit includes disassociating the sample into a dissociate of single intact cells.

18. The laboratory sample dissociation method of claim 16, wherein loading a sample and a buffer includes loading a sample and a buffer into a tube assembly so that the sample and the buffer occupy 20 percent to 90 percent of the tube chamber.

19. The laboratory sample dissociation method of claim 16, wherein operating the processing unit includes operating at a speed of 0.8 to 1.6 m/s, for a time of 30 sec to 60 sec, or both.

20. The laboratory sample dissociation method of claim 16, further comprising directly placing the processed tube assembly into a centrifuge without transferring the dissociated sample to a separate tube assembly; and spinning down the dissociated sample using the centrifuge to separate out single intact cells from extracellular debris.
